(19) United States
(12) Patent Application Publication
(10) Pub. No.: US 2025/0179409 A1
(43) Pub. Date: Jun. 5, 2025

(54) CELL PROLIFERATION APPARATUSES AND USES THEREOF

(71) Applicant: DUOGENIC STEMCELLS CORPORATION, Taichung City (TW)

(72) Inventors: Masataka KIYOKAWA, Kyoto (JP); Hong-Lin SU, Taichung City (TW); Ching-I SHEN, Taichung City (TW); Fu-Hui WANG, Taichung City (TW); Chia-Ying HSIEH, Taichung City (TW)

(21) Appl. No.: 18/840,734

(22) PCT Filed: Feb. 24, 2023
(86) PCT No.: PCT/JP2023/006684

§ 371 Date: Aug. 22, 2024

(30) Foreign Application Priority Data
Feb. 25, 2022 (AU) 2022900445

(51) Int. Cl. C12M 1/00; C12M 1/04

(52) U.S. Cl. CPC C12M 41/14; C12M 23/14; C12M 23/24; C12M 29/00

(57) ABSTRACT

Provided is a cell proliferation apparatus, comprising (a) a device for cell storage or cell culture; and (b) a means or a unit to deliver an airflow to the device for cell storage or the cell culture. Also provide is an in vitro method to induce cell proliferation, by placing a plurality of cells in the cell proliferation apparatus described herein.

### CROSS-REFERENCES TO RELATED APPLICATION

This application claims the benefit of priority from Australian Provisional Application No. 2022900445, filed 25 Feb. 2022, the entire content of which is incorporated herein by reference.

### FIELD

The invention relates to cell proliferation apparatuses. Also provided are methods for growing cells in vitro using the cell proliferation apparatuses disclosed herein.

### BACKGROUND

Cell culture refers to a process that allows cells obtained from animals, humans and plants to grow outside their natural environment. Cell culture using various cell culture devices is the mainstay of experimental cell biology and cell-based therapies targeting multiple systems. The interplay between cell culture parameters and the microenvironmental conditions in the cell culture device can significantly affect the number of cell harvest as well as the in vivo performance of the cultured cells.

Commercial and investigational cell cultures are increasingly manufactured using cell culture devices, such as a cell culture bag, which decreases risks of contamination and facilitates scale-up and automation. However, the full potential and efficacy of the cell culture devices for large-scale manufacturing of cells are still yet to be optimized, especially for cell therapy applications.

There is a need for a more efficient cell proliferation apparatus that enable cost-effective and consistent manufacturing of high-quality cells at large-scale to meet the regulation of good manufacturing practice (GMP) for advanced therapy medicinal products (ATMP), such as autologous and allogeneic cell therapy with or without genetic modification. The present invention addresses this need and other needs.

### SUMMARY OF THE INVENTION

In one embodiment, the present invention provides a cell proliferation apparatus, comprising (a) a device for cell storage or cell culture; and (b) a mean or a unit for delivering an airflow to the device for cell storage or the cell culture, wherein the direction of the airflow is from the mean or the unit directly to the surface of the device with over 50% of the cells resting thereupon (hereafter, surface of the device). The rate of cell proliferation increases with the speed of airflow compared to that of a cell proliferation apparatus without a mean or a unit to deliver any airflow.

In an exemplary embodiment, the angle between the means to deliver the airflow and the surface of the device with over 50% of the cells resting thereupon (see * in FIG. 8A, hereafter fan angle) is greater than 45° to about 90°. In another exemplary embodiment, % of surface of the device (with over 50% of the cells resting thereupon), that is blown by or in contact to the wind or airflow, is at least 50%.

Some embodiments provide in vitro methods to induce cell proliferation by placing a plurality of cells in the device for cell storage or cell culture of the cell proliferation apparatus described herein.

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

The invention will become more apparent when read with the accompanying figures and detailed descriptions which follow.

### BRIEF DESCRIPTION OF DRAWINGS

Illustrative embodiments of the present invention are described in detail below with reference to the following Figures:

FIG. 1 shows an exemplary embodiment of the invention comprising a device for cell culture or cell storage (A), a mean or a unit to deliver an airflow to the device (B) and a housing (C). The white arrows indicate the direction of airflow and the black arrow indicates the surface of the device for cell storage or cell culture with over 50% of the cells resting thereupon.

FIG. 2A is a line graph and FIG. 2B is a bar graph illustrating the cell number and fold increase of Jurkat cells cultured in the cell proliferation apparatus of the present invention (w/fan at an airflow speed of 0.82 m/s) and in a cell culture device without a fan or airflow (w/o fan) (*p<0.05, **p<0.01, and ***p<0.001).

FIG. 3A is an assembly of flow cytometric analysis images showing similar CD3 expression levels of Jurkat cells cultured in the cell proliferation apparatus of the present invention (w/fan at an airflow speed of 0.82 m/s) and in a cell culture device without a fan or airflow (w/o fan). FIG. 3B is an assembly of flow cytometric analysis images showing the % of cell death of Jurkat cells cultured in the cell proliferation apparatus of the present invention (w/fan at an airflow speed of 0.82 m/s) and in a cell culture device without a fan or airflow (w/o fan).

FIG. 4A is a line graph and FIG. 4B is a bar graph illustrating the effect of various airflow speeds on the cell number and fold increase of Jurkat cells cultured in the cell proliferation apparatus of the present invention (to deliver an airflow speed of 0.46, 0.82, and 1.19 m/s) and in a cell culture device without a fan or airflow (*p<0.05, **p<0.01, and ***p<0.001).

FIG. 5 is a line graph illustrating the cell number of human peripheral blood mononuclear cells (PBMNCs) cultured in the cell proliferation apparatus of the present invention (w/fan to deliver an airflow speed of 0.82 m/s) and in a cell culture device without a fan (w/o fan).

FIG. 6 is an assembly of photo images showing the cell proliferation of the present invention (w/fan, at an airflow speed of 0.82 m/s) does not increase the risk of bacteria penetration through the cell culture bag.

FIG. 7A to FIG. 7C are line graphs illustrating the cell number and fold increase of Jurkat cells cultured in the cell proliferation apparatus of the present invention (w/fan at an airflow speed of 1.19 m/s). In FIG. 7A, the cells were cultured in a cell culture bag with a bag wall thickness of 0.076±0.013 mm. In FIG. 7B, the cells were cultured in a cell storage bag with a bag wall thickness of 0.406±0.005 mm. In FIG. 7C, the cells were cultured in a cell storage bag with a bag wall thickness of 0.473±0.127 mm.

FIG. 8A to FIG. 8C are graphs illustrating the fan angle (“*” in FIG. 8A) between (a) the direction of airflow from a fan and (b) the surface of the device for cell storage, said surface is where >50% of the cultured cells resting thereupon; and the effect of fan angels on the proliferation of Jurkat cells cultured in the cell proliferation apparatus of the present invention (with a fan to deliver an airflow speed of 1.19 m/s). FIG. 8A illustrates the various fan angles. FIG. 8B and FIG. 8C are bar graphs showing the effect of various fan angles (horizontal angle) (0°), vertical angle (90°)and 45° angle) on cultured cell number.

FIG. 9 is a bar graph illustrating the effects of surface area exposed to an airflow on cultured cell number in the cell proliferation apparatus of the present invention (with a fan to deliver an airflow speed of 1.19 m/s).

### DETAILED DESCRIPTION OF THE INVENTION

As used herein, the articles “a” and “an” refer to one or more than one (i.e., at least one) of the grammatical object of the article. By way of example, “a fan” means one fan or more than one fan.

The term “culture” refers to a process that allows cells or tissues obtained from animals, humans and plants to grow outside their natural environment.

The term “cell” includes adherent and non-adherent cells. The non-adherent cells include nucleated cells, non-nucleated cells and platelets.

All numbers herein are understood as modified by “about.” As used herein, the term “about” is meant to encompass variations of ±10%.

### Culture Proliferation Apparatus

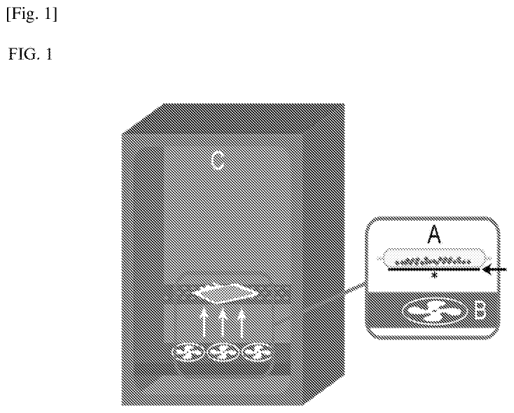

As shown in FIG. 1, the present culture proliferation apparatus comprises (i) a device for cell culture or cell storage (A); and (ii) a mean or a unit to deliver airflow to the device for cell storage or the cell culture (B), wherein the direction of the airflow is from the mean or the unit directly to the surface of the device with over 50% of the cells resting thereupon (the direction of airflow is illustrated by the white arrows and the surface of the device for cell storage or cell culture with over 50% of the cells resting thereupon is illustrated by * and the black arrow).

The present culture proliferation apparatus is superior to a similar culture platform without the mean or the unit to deliver an airflow to the device surface (wherein >50% resting thereupon), by increasing the rate of cell proliferation and reducing the rate of cell death, yet retain the characteristics of the cultured cells.

In one embodiment, the surface of the device for cell storage or cell culture includes over 50%, 55%, 60%, 65%, 70%, 75%, 80%, 85%, 90%, 95% or 100% of cultured cells resting thereupon. In another embodiment, the surface of the device for cell storage or cell culture includes about 50%-100%, about 55%-100%, about 60%-100%, about 65%-100%, about 70%-100%, about 75%-100%, about 80%-100%, about 85%-100%, about 90%-100% or about 95%-100% of cultured cells resting thereupon.

In some embodiments, the culture proliferation apparatus further comprises a housing (C) as illustrated in FIG. 1. In other embodiments, the culture proliferation apparatus is free of a heater or a dehumidifier. In other embodiments, the culture proliferation apparatus comprises a heater or a dehumidifier

In an embodiment, a portion of the device for cell culture or cell storage is gas permeable. In another embodiment, a portion of the device for cell culture or cell storage has gas permeability of 3 to 1000 barrers or any value or range of values therebetween in 1 barrer increments (e.g., about 3 barrers to 40 barrers, about 3 barrers to about 50 barrers, about 3 to 100 barrers, about 3 to 200 barrers, about 3 to 300barrers, about 3 to 400 barrers, about 3 to 500 barrers, about 3 to 600 barrers, about 3 to 700 barrers, about 3 to 800 barrers, about 3 to 900 barrers, about 4 to 1000 barrers, about 4 to 40 barrers, about 5 to 1000 barrers, about 5 to 40 barrers).

In some embodiments, the device for cell culture is a bag, a plate, a dish, a flask, a bottle, one or more wells, or a bioreactor. In some embodiments, the device for cell culture is a device disclosed in WO2005035728 (the content of which is incorporated by reference), comprising a top and a bottom joined by a plurality of sides, wherein the bottom comprises a gas permeable material, and at least a portion of a side is comprised of a gas permeable material, such that cells can be cultured when said apparatus is oriented in at least two positions, vertically and horizontally.

The thickness of the cell culture bag is about 0.01 mm to about 0.2 mm or any value or range of values therebetween in 0.01 mm increments (e.g., about 0.01 to about 0.19 mm, about 0.05 to about 0.15 mm etc.).

In one aspect of the invention, the device for cell storage is a cell storage bag. The thickness of the cell storage bag is about 0.2 mm to about 0.5 mm or any value or range of values therebetween in 0.01 mm increments (e.g., about 0.2 mm to about 0.35 mm, about 0.2 to about 0.40 mm, about 0.2 to about 0.45 mm, about 0.2 mm to about 0.47 mm etc.).

In another aspect of the invention, a device for cell storage or cell culture comprises a basal medium with suitable supplements for cell culture. Non-limiting examples of the basal medium are: basal medium eagle, αMEM, GMEM, DMEM, DMEM/F12, RPMI-1640, IMDM, MCDB series, modified basal mediums, etc. Non-limiting examples of the supplements are: fetal bovine serum, fetal calf serum, human serum, human platelet lysates, albumins, pituitary gland extracts, tissue extracts, defined or non-defined chemical supplements, etc.

Non limiting examples of the mean or the unit to deliver the airflow is a fan, a wind blowing device or an electrostatic air mover.

The distance between the mean or the unit to deliver the airflow and the surface of the device with over 50% of the cells resting thereupon is about 0.5 cm to about 35 cm, or any value or range of values therebetween in 0.5 cm increments (e.g., about 1 to about 25 cm, about 1 to about 20 cm, about 1 to about 30 cm, etc.). In an exemplary embodiment, there is no object to obstruct the flow of the airflow or wind between the mean or the unit to deliver the airflow and the surface of the device.

The speed of the airflow (from the device), measured at * of FIG. 1, where the surface of the device with over 50% of the cells resting thereupon, is 0.01 m/s to 20 m/s or any value or range of values therebetween in 0.1 cm increments (e.g., about 0.3 to 2.0 m/s, about 0.1 m/s to 2 m/s, about 0.1 m/s to 3 m/s, about 0.1 m/s to 4 m/s, about 0.1 m/s to 5 m/s, about 0.1 m/s to 15 m/s, about 0.05 m/s to 10 m/s, etc.).

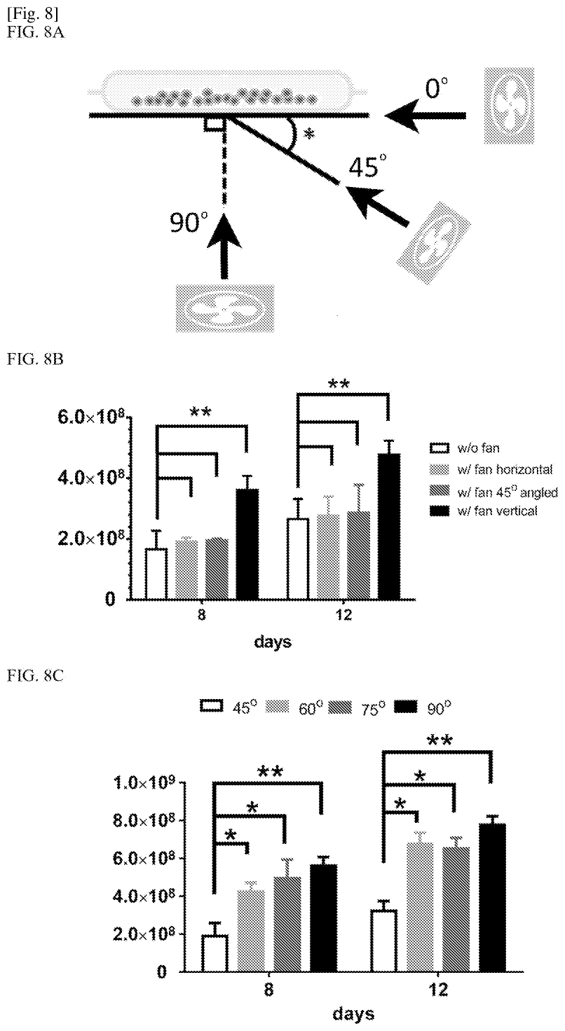

FIG. 8A illustrates the fan angle (*), which is the angle between (a) the direction of airflow generated by a mean or a unit to deliver an airflow; and (b) the surface of the device for cell storage or cell culture, said surface is where >50% of the cultured cells resting thereupon. In one embodiment, the fan angle is greater than 45° to about 90°,about 50° to about 90°, about 55° to about 90°, about 60° to about 90°, about 65° to about 90°, about 70° to about 90°, about 75° to about 90°, about 80° to about 90° and about 85° to about 90°.

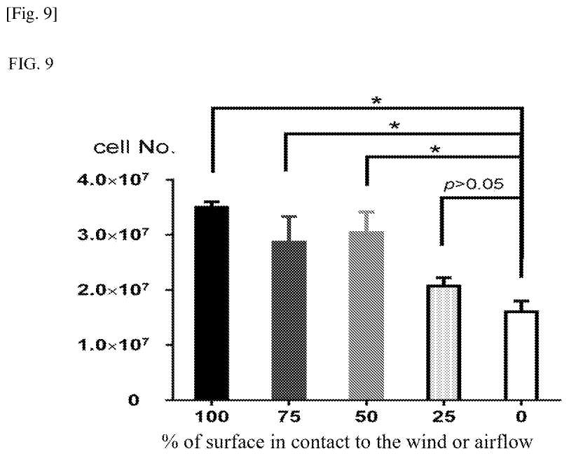

As illustrated by FIG. 9, the % of surface of the device (with over 50% of the cells resting thereupon), that is blown by or in contact to the wind or airflow generated by the means or the unit to deliver an airflow, also affects the efficiency of cell culture. In an exemplary embodiment, the % of surface of the device with over 50% of the cells resting thereupon (a black arrow and * in FIG. 1) that is blown by or in contact to the wind or airflow, is greater than 25%, 30%, 35%, 40%, 45%, 50%, 55%, 60%, 65%, 70% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% to 100% or any range therein between, for example, about 25% to 100%, about 30% to 100%, about 35% to 100% and about 40% to 100%, about 45% to 100%, about 50% to 100%, about 55% to 100%, about 60% to 100%, about 65% to 100% and about 70% to 100%, about 75% to 100%, about 80% to 100%, about 85% to 100%, about 90% to 100% and about 95% to 100%.

### In Vitro Culture Methods for Inducing Cell Proliferation

The invention also provides an in vitro method to induce cell proliferation, comprising the steps of
(a) placing a plurality of cells in a device for cell storage or cell culture described herein, and
(b) delivering an airflow by a mean or a unit to the device in step (a),

wherein the direction of the airflow is from the unit directly to the surface of the device with over 50% of the cells resting thereupon.

In an exemplary embodiment, and the angle between the means to deliver the airflow and the surface of the device with over 50% of the cells resting thereupon is greater than 45° to about 90°. In another exemplary embodiment, the % of surface of the device (with over 50% of the cells resting thereupon), that is blown by or in contact to the wind or airflow also affects the efficiency of cell culture. In an exemplary embodiment, the % of surface of the device with over 20% of the cells resting thereupon (a black arrow and * in FIG. 1) that is blown by or in contact to the wind or airflow, is greater than 25% to 100%.

The in vitro method provided herein, by placing the plurality of cells in the cell proliferation apparatus with an airflow directly to the surface of the device with over 50% of the cells resting thereupon, unexpectedly increase the rate of cell proliferation and reduce the rate of cell death compared to placing the plurality of cells in a cell proliferation device without an airflow. Furthermore, the rate of cell proliferation increases with the increase of airflow speed.

The following examples of specific aspects for carrying out the present invention are offered for illustrative purposes only, and are not intended to limit the scope of the present invention in any way. The airflow speed mentioned in the following examples is measured at the surface of the culture bag where more than 50% of the cultured cells rest on.

### Examples

### Example 1

### The Effect of Airflow on Cultured Jurkat Cells

The Jurkat cells, human T cell leukemia cells, were seeded in CultiLife™ 215 culture bag (commercially available from Takara, Japan) at a concentration of 1×10⁶ cells/mL in 50 mL RPMI-1640 with 10% fetal bovine serum (FBS) and cultured in a 37° Cincubator with a humidified atmosphere of 5% CO₂, with or without a fan. The fan delivered an airflow speed of 0.82 m/s. The culture medium was refreshed by adding an equal volume of original culture medium at the interval of two days. The final volume of culture medium was 200 mL per bag.

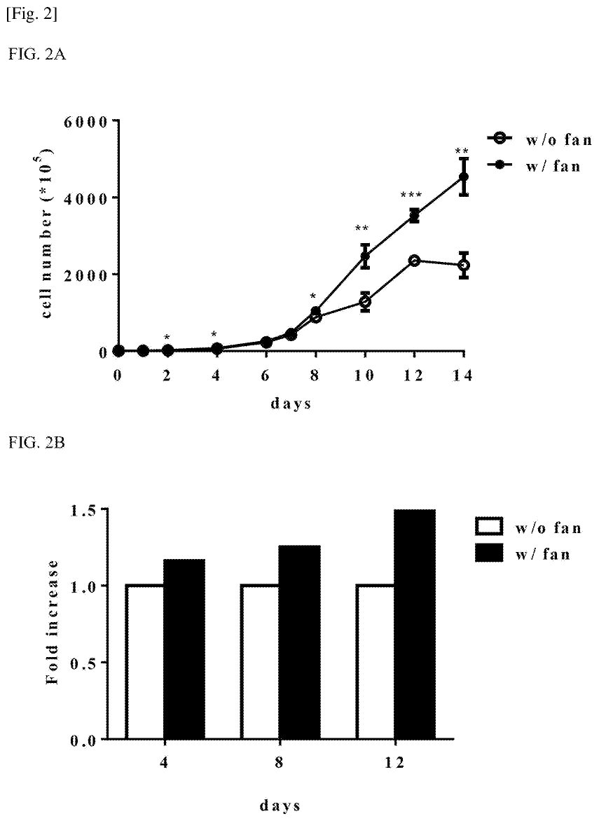

FIG. 2 shows the significant increase of the number and fold increase of cultured Jurkat cells with the use of a fan to provide airflow compared to without using a fan.

### Example 2

### The Effect of Airflow on Characteristics and Survival of Cultured Jurkat Cells

The Jurkat cells cultured with or without a fan from Example 1 were stained with anti-human CD3-APC (1 ng/mL, BioLegend, USA) and propidium iodide (PI, 10 ng/mL). Cultured Jurkat cell suspension was prepared and adjusted to a concentration of 1×10⁷ cells/ml with cold PBS-BSA (phosphate-buffered saline with bovine serum albumin). Aliquot 100 μl of the cell suspension into the required number of tubes. Antibody was added at the vendor-recommended dilution and mixed thoroughly and incubated at 4° C. for at least 30 minutes. Cells were washed with 1 ml of cold PBS-BSA and centrifuged at 400 g for 5 minutes. The supernatant was discarded and cells were resuspended in 300-500 μl PI (propidium iodide, 10 ng/ml)/Triton X-100 (0.3%) staining solution at 37° C. for 15 minutes or at 20° C. for 30 min. The tubes were transferred to ice or stored at 4° C. protected from light. The cultured cells were analyzed by Flow Cytometry.

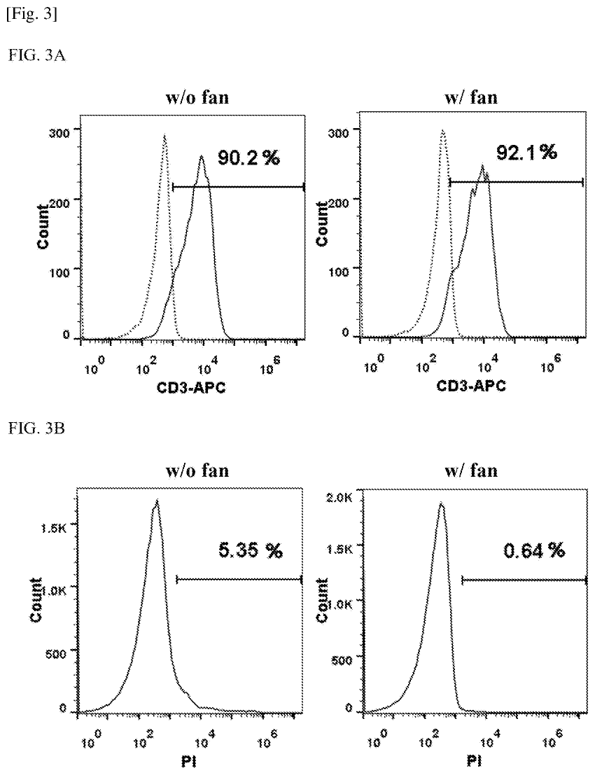

The cells cultured with or without fan showed similar expression levels of CD3, suggesting that the phenotype of the T cells were maintained with fan (FIG. 3A). However, an increased cell death was noted in cells cultured without a fan (5.35%) compared to cells cultured with a fan (0.64%), illustrated by PI staining. See FIG. 3B.

### Example 3

### The Effect of Airflow Speed on the Culture Jurkat Cells

The Jurkat cells were seeded in CultiLife™ 215 Culture Bag at a concentration of 1×10⁶ cells/mL in 5 0mL RPMI-1640 with 10% fetal bovine serum (FBS) and cultured in a 37° C. incubator with humidified atmosphere of 5% CO₂, with a fan at different airflow speeds (0.46, 0.82, and 1.19 m/s) or without a fan. The culture medium was refreshed by adding equal volume of original culture medium at the interval of two days. The final volume of culture medium was 200 mL per bag.

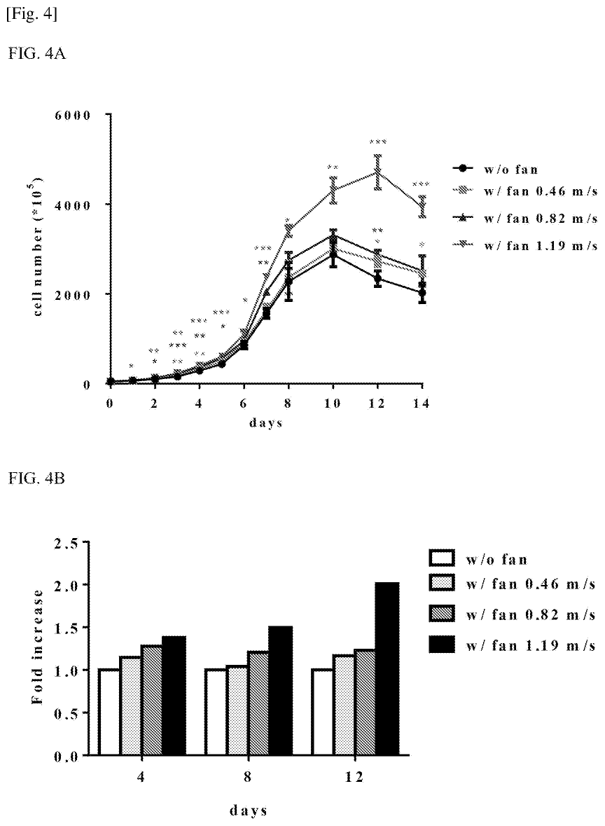

FIG. 4A and FIG. 4B show that the rate of cell proliferation increases as the airflow speed increases.

### Example 4

### The Effect of Airflow on Cultured Human Peripheral Blood Mononuclear Cells (PBMNCs)

A blood sample (20 mL) was diluted to a 1:1 volume ratio with PBS and a 2 volume of peripheral blood mononuclear cells (PBMNCs) separation solution (Ficoll-paque 1.077, Cytiva) was added to a fresh tube. Diluted blood was gently layered on top of the Ficoll to avoid mixing together. The tube was centrifuged at 800×g for 20-30 minutes. The mononuclear cells were harvested to a new tube and were washed twice with PBS. The PBMNCs were collected and cultured in a cell storage bag (JMS, Japan) comprising DuoCIK media at 37° C. with a humidified atmosphere of 5% CO₂, in the cell proliferation apparatus of the present invention (with a fan at an airflow speed of 1.19 m/s) and in a cell culture device without a fan.

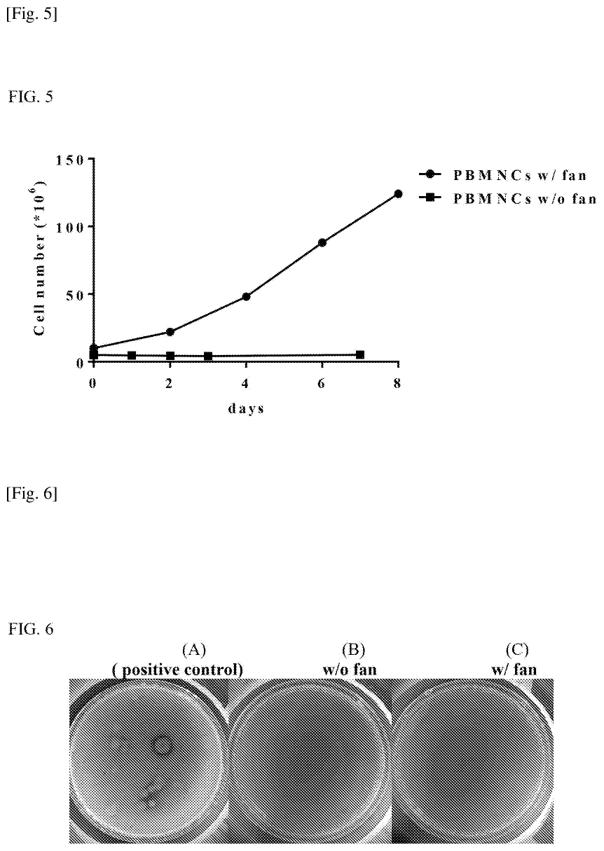

FIG. 5 shows the number of cultured human PBMNCs increases in the presence of a fan to deliver airflow to the cell culture bag, rather than in the bag without a fan.

### Example 5

### The Effect of Airflow on Microorganism Permeability of Cell Culture Bag

A cell storage bag (JMS, Japan) was filled with 200 mL of LB medium and cultured in a 37° C. incubator with a fan to deliver an airflow speed of 0.82 m/s or without a fan. To illustrate the medium in the cell culture bag can support bacterial growth, 100 μL of E. coli in LB medium (strain DH5α, OD 0.4) was added to the cell culture bag containing 200 mL LB medium. After 1 day culture in a 37° C. incubator, the LB medium (with added bacteria) was spread on LB agar plates and colony formation was observed after one day culture of culture (panel A of FIG. 6). The LB medium (without added bacteria) was collected from the cell culture bag with or without a fan and spread on LB agar plates. No colony formation was observed after 30 days of culture, indicating the airflow to the cell culture bag does not affect the impermeable nature of the cell culture bag to environmental microbes (panels B and C of FIG. 6).

### Example 6

### The Effect of Airflow on Cell Culture Bag and Cell Storage Bag

The Jurkat cells were seeded in cell culture bag 1 (CultiLife™ 215 Culture Bag) or cell storage bags 2 & 3 (bag 2, cell storage bag, commercially available from JMS, Japan; bag 3, cell storage bag, commercially available from Kawasumi, Japan) at a concentration of 1×10⁶ cells/mL in 50 mL of RPMI-1640 with 10% fetal bovine serum (FBS). The cells were cultured in a 37° C. incubator with humidified atmosphere of 5% CO₂ with a fan to deliver an airflow speed of 1.19 m/s or without a fan.

The thickness of cell culture bag 1 was 0.076±0.013 mm, cell storage bag 2 was 0.406±0.005 mm, and cell storage bag 3 was 0.473±0.127 mm.

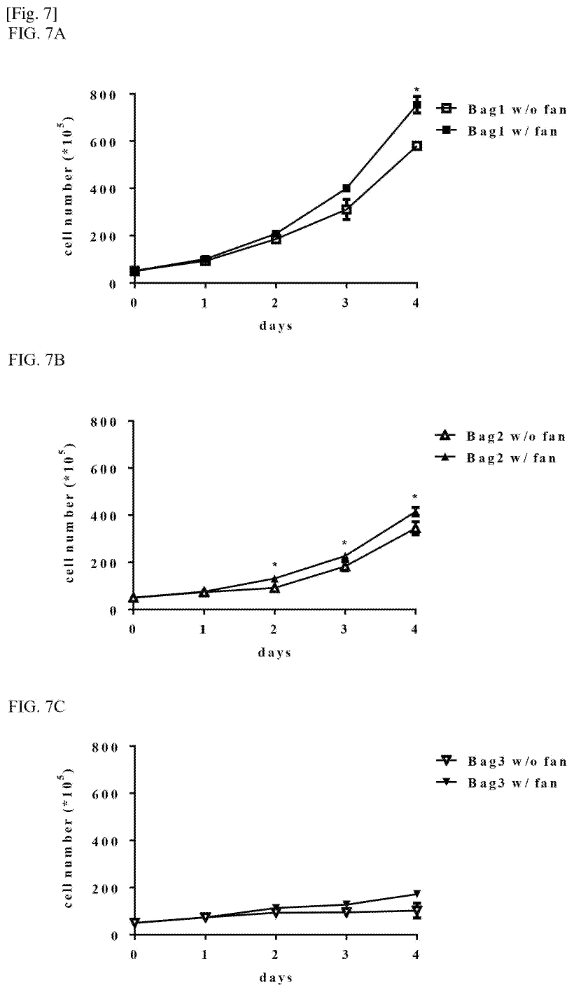

FIG. 7A to FIG. 7C show the number of cultured cells increases with the use of fan, in both cell culture bag and cell storage bag.

### Example 7

### The Effect of Fan Angles on Cell Proliferation in Cell Storage Bag

The Jurkat cells were seeded in cell storage bags according to the steps in Example 1, with a fan to deliver an airflow speed of 1.19 m/s at various fan angles (0°, 45° and) 90° or without a fan.

FIG. 8B and FIG. 8C show the effect of fan angles on cell proliferation. The cultured cell number increases as the fan angle increases. Specifically, the cell proliferation significantly increase as the fan angle is greater than 45°. *. p<0.05. **, p<0.01.

### Example 8

### The Effect of Surface Area Permeable to or in Contact With Airflow on

### Cell Proliferation in Cell Storage Bag

The Jurkat cells were seeded in cell storage bags according to the steps in Example 1 with a fan to deliver an airflow speed of 1.19 m/s or without a fan.

The surface of the cell storage bag, where over 50% of the cells resting thereupon (see * in FIG. 1), was partially covered by a plastic wrap so that 25%, 50% and 75% of the surface is covered and impermeable to the wind or breeze generated by a fan or not covered. This means 75%, 50%, and 25% or 100% (not covered) of the surface of the cell storage bag was in contact with the wind or breeze generated by a fan, respectively.

FIG. 9 shows the cell number of Jurkat cells cultured in the cell proliferation apparatus of the present invention increases as the % of the surface permeable to the wind or breeze is greater than 25%.

1. A cell proliferation apparatus, comprising
(a) a device for cell storage or cell culture; and
(b) a means to deliver an airflow to the device for cell storage or the cell culture,
wherein the direction of the airflow is from the means directly to the surface of the device with over 50% of the cells resting thereupon and the angle between the means to deliver the airflow and the surface of the device with over 50% of the cells resting thereupon is greater than 45° to about 90°.

2. The cell proliferation apparatus of claim 1, wherein the device for cell storage is a cell storage bag.

3. The cell proliferation apparatus of claim 1, wherein a portion of the device for cell culture is gas permeable.

4. The cell proliferation apparatus of claim 1, wherein the device for cell culture is a bag, a plate, a dish, a flask, a tube, a well or a bioreactor.

5. The cell proliferation apparatus of claim 1, wherein the device for cell storage or cell culture comprises a medium.

6. The cell proliferation apparatus of claim 1, wherein the speed of the airflow is between 0.03 m/s to 20 m/s at the surface of the device where over 50% of the cells resting thereupon.

7. The cell proliferation apparatus of claim 1, wherein the means to deliver the airflow is a fan or an electrostatic air mover.

8. The cell proliferation apparatus of claim 1, wherein the distance between the means to deliver the airflow and the device is about 1 cm to about 20 cm.

9. The cell proliferation apparatus of claim 1, wherein the apparatus is free of a heater or a dehumidifier.

10. A cell proliferation apparatus, comprising.
(a) a device for cell storage or cell culture; and
(b) a means to deliver an airflow to the device for cell storage or the cell culture,
wherein the direction of the airflow is from the means directly to the surface of the device with over 50% of the cells resting thereupon and greater than 25% to 100% of the surface of the device is in contact to the airflow generated by the means.

11. An in vitro method to induce cell proliferation, comprising the steps of:
(a) placing a plurality of cells in a device for cell storage or cell culture of claim 1; and
(b) delivering an airflow by a unit to the device in step (a),
wherein the direction of the airflow is from the unit directly to the surface of the device with over 50% of the cells resting thereupon.

12. The in vitro method of claim 11, wherein the device for cell storage is a cell storage bag.

13. The in vitro method of claim 11, wherein a portion of the device for cell culture is gas permeable.

14. The in vitro method of claim 14, wherein the device for cell culture is a bag, a plate, a dish, a flask, a tube, a well or a bioreactor.

15. The in vitro method of claim 14, wherein the device for cell storage or cell culture comprises a medium.

16. The in vitro method of claim 14, wherein the speed of the airflow is between 0.03 m/s to 20 m/s at the surface of the device where over 50% of the cells resting thereupon.

17. The in vitro method of claim 14, wherein the unit to deliver the airflow is a fan or an electrostatic air mover.

18. The in vitro method of claim 14, wherein the distance between the unit and the device is about 1 cm to about 20 cm.
